(19) United States
(12) Patent Application Publication
(10) Pub. No.: US 2005/0278037 A1
(43) Pub. Date: Dec. 15, 2005

(54) Implant for the treatment of cystocele and rectocele

(75) Inventors: Emmanuel Delorme, Chalon Sur Saone (FR); Jean-Marc Beraud, St. Etienne (FR)

Correspondence Address: DENNISON, SCHULTZ, DOUGHERTY & MACDONALD, 1727 KING STREET, SUITE 105, ALEXANDRIA, VA, 22314, US

(73) Assignee: ANALYTIC BIOSURGICAL SOLUTIONS-ABISS

(21) Appl. No.: 11/146,280

(22) Filed: Jun. 7, 2005

Related U.S. Application Data
(60) Provisional application No. 60/607,267, filed on Sep. 7, 2004.

(30) Foreign Application Priority Data
Jun. 11, 2004 (FR) 04 06352

(51) Int. Cl. A61F 2/02

(52) U.S. Cl. 623/23.72; 600/37; 606/151

(57) ABSTRACT

Implant for the treatment of cystocele, rectocele and/or prolapse of the vaginal dome, with a thin, flexible structure including a support body (2) starting from which extend at least:
two upper suspension stabilisers (3),
and two lower suspension stabilisers (4) on each side of the sagittal plane (S),
   characterised in that each suspension stabiliser (3, 4) is made so that its extensibility parallel to its longitudinal axis is low.

The technical domain of this invention is cystocele and rectal prolapses, particularly in elderly women.

Prolapse phenomena usually result from relaxation of the genital or rectal organ suspension tissues leading to problems that require a surgical operation.

Thus, attempts were proposed to reconstruct the natural suspension system of organs affected by the prolapse, by using nonabsorbable sutures or reinforcement bands. However, these techniques have not always been satisfactory, particularly due to the need for a major surgical operation leading to a dissection of regions of the anatomy not concerned by the surgical repair, to make nonabsorbable sutures.

In an attempt to overcome these disadvantages, a patent application FR 2 785 521 proposed to use an implant comprising a support body from which two suspension bands extend, fitted at their ends with anchorage parts designed to be sutured in areas known as being anatomically stable. This implant was then implanted using a laparoscopic method to simplify the surgical procedure.

However, it was found that such an implant cannot be efficiently suspended, particularly due to stresses applied at areas known as being anatomically stable. Furthermore, this type of implant does not have very good spatial stability under usage conditions.

Thus, the need arises firstly to have an implant with better implantation stability and secondly a technique for obtaining optimum stability while minimising trauma suffered by the patient.

Thus, in order to achieve these objectives, an implant is proposed for the treatment of rectocele or cystocele, with a thin and flexible structure and comprising a support body from which at least two upper suspension stabilisers extend on each side of a sagittal plane of the support body and two lower suspension stabilisers also arranged on each side of the sagittal plane.

This new implant is fully satisfactory, however it was found that it could be further improved. It was found that in some usage configurations, a high tension applied to the suspension stabilisers induced an undesirable and uncomfortable deformation of the said suspension stabilisers such that it is difficult to quickly place the stabilisers without some residual tension or to maintain an appropriate configuration of the body of the implant or to control the conformation of the implantation of the implant or prosthesis assembly.

In order to provide a solution to this problem, the invention relates to an implant for the treatment of the cystocele, rectocele and/or prolapse of the vaginal dome, with a thin, flexible structure including a support body starting from which extend at least:
two upper suspension stabilisers,
and two lower suspension stabilisers on each side of the sagittal plane.

According to the invention, this implant is characterised in that each suspension stabiliser is made so that its extensibility parallel to its longitudinal axis is low.

The inventors have determined that accidental deformations of the implant and particularly the suspension stabilisers could be caused by the mechanical properties of the material from which the implant is made, particularly when the implant is cut out of a single piece of woven or knitted biocompatible material such that the centre line of the suspension stabilisers is not parallel to either of the preferred directions of tensile strength.

The inventors also determined that the best behaviour of the implant is obtained when the support body is made from a relatively flexible and extensible material, while the suspension stabilisers on which the surgeon applies the tension force are made from a slightly extensible prosthetic material in order to limit their deformation so that they can maintain a “flat” conformation and thus enable the surgeon to adjust the assembly “tension” more accurately than with a prosthetic material that is too elastic.

Thus, according to one preferred but not strictly necessary characteristic when manufacturing the invention, the extensibility of suspension stabilisers parallel to their longitudinal axis is less than the extensibility of the body of the implant particularly along its two longitudinal and transverse axes.

According to the invention, there are different ways of obtaining these differences in behaviour of the support body and suspension stabilisers under tension.

Thus, according to one embodiment, the implant assembly conforming with the invention is knitted in a single piece, but a different reinforcement and stitch orientation is adopted for the body and the suspension stabilisers, such that the longitudinal extensibility of the suspension stabilisers is low and is preferably lower than the longitudinal extensibility of the implant body.

According to another embodiment, this differential behaviour is obtained by adding each suspension stabiliser onto the support body. It is thus possible to orient the constituent material of each suspension stabiliser so as to obtain the required mechanical behaviour.

According to one characteristic of the invention, the support body has an attachment tab for each suspension stabiliser, to facilitate attachment of the suspension stabilisers onto the support body.

The placement and attachment of the suspension stabilisers can then be achieved in different ways.

According to one characteristic of the invention, each suspension stabiliser is sewn onto the support body.

According to another characteristic of the invention, each suspension stabiliser is glued onto the support body.

According to yet another characteristic of the invention, each suspension stabiliser is welded onto the support body.

Obviously, these assembly modes could be combined to make implants according to the invention.

According to the invention, each suspension stabiliser may be made from different types of biocompatible materials that may or may not be absorbable, synthetic or of animal and/or vegetable origin. The biocompatible synthetic materials that could be used include polyester, polypropylene and polyamides. Similarly, biocompatible materials with animal origin include transformed pork collagen.

The material from which suspension stabilisers are made may then have different types of structures, for example it may be composed of woven or knitted threads or fibres, or fibres assembled to form a non-woven fibrous material. The material from which the suspension stabilisers are made could also be formed from a film or a complex of films made from biocompatible materials.

According to one embodiment, each suspension stabiliser is made from a knitted material for which the weft threads or the warp threads are arranged parallel to the longitudinal axis of the suspension stabiliser.

Preferably, each suspension stabiliser is then made from a slightly extensible knit parallel to the longitudinal axis of the suspension stabiliser.

In another embodiment, each suspension stabiliser is made from a layer of non-woven material with an isotropic tensile strength in its extension plane.

According to the invention, the body of the implant support may be in different shapes, for example such as a triangular, oval or even circular shape. In one preferred but not exclusive embodiment, the support body has a generally rectangular shape with a length between 60 and 90 mm and a width between 40 and 60 mm, although these dimensions are in no way limitative.

Obviously, different conformations or configurations could be envisaged for suspension stabilisers and the support body according to the invention.

Thus, in one embodiment, the longitudinal axes of the upper stabilisers form an angle of more than 45° and preferably but not strictly necessarily an angle α between 100° and 180° and preferably between 115° and 170°. It should be noted that according to one preferred embodiment, the sagittal plane forms an axis of symmetry of the implant and therefore bisects the angle α.

When upper suspension stabilisers with such a relative orientation are used, they can be judiciously placed in the trans-sacrosciatic region to give a good distribution of forces applied to the support body at the anatomic anchorage points of the stabilisers, while guaranteeing the best spatial orientation of the support body implanted in the patient.

According to one preferred but not strictly necessary characteristic of the invention, the length of the upper and/or lower suspension stabilisers is greater than 100 mm and preferably greater than or equal to 120 mm. This length enables good extension of the suspension stabilisers in their corresponding insertion areas and takes advantage of friction between the suspension stabilisers or arms and the tissues passed through to maintain the implant.

According to another characteristic of the invention, the longitudinal axes of the lower stabilisers preferably but not necessarily form a non-zero angle β. Thus, it must be considered that the lower stabilisers are not parallel to each other. The angle β is preferably, but not strictly necessary, greater than 10° so that it is preferably between 10° and 75°, or between 100° and 180° depending on the pathology to be treated.

According to yet another characteristic of the invention that is preferred but not strictly necessary, the support body has an approximately rectangular general shape. Preferably but not strictly necessary, the support body is between 60 mm and 90 mm and between 40 mm and 60 mm wide.

According to one embodiment of the invention, the upper stabilisers extend approximately from the upper corners of the support body and the lower stabilisers extend from the lower corners of the support body.

According to another embodiment of the invention, the upper stabilisers extend approximately from the upper corners of the support body, while the lower stabilisers extend from the two large sides of the support body. Each of the lower stabilisers then preferably but not necessarily extends to a distance from the upper edge of the support body equal to between 60% and 87% of the length of the support body.

According to another characteristic of the invention, the lower stabilisers have a widened area at their connection with the support body of the implant.

Various other characteristics of the invention will become clear from the following description made with reference to the attached drawings that illustrate different embodiments of an implant according to the invention, and insertion devices used to facilitate placement of the said implant.

It should also be noted that the different characteristics of the invention described above and below can be combined in different variants depending on the pathology to be treated.

FIG. 1 is an elevation showing a flat view of an implant according to the invention designed for the treatment of rectocele.

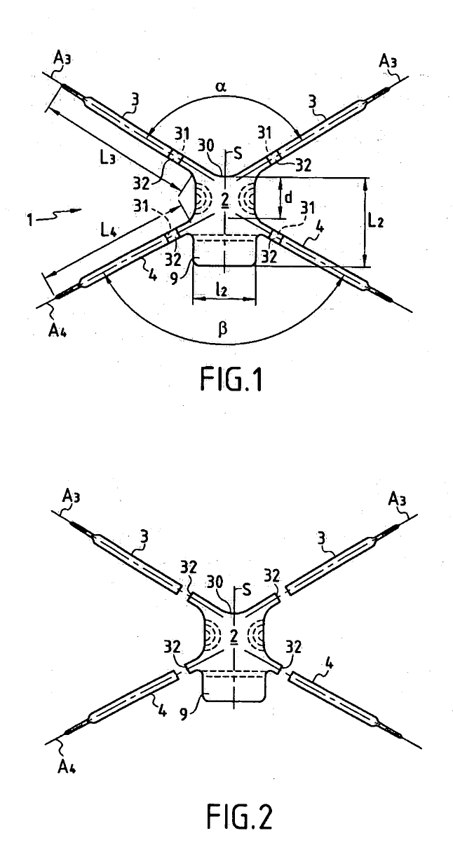

FIG. 2 is an elevation showing an exploded flat view of the implant shown in FIG. 1.

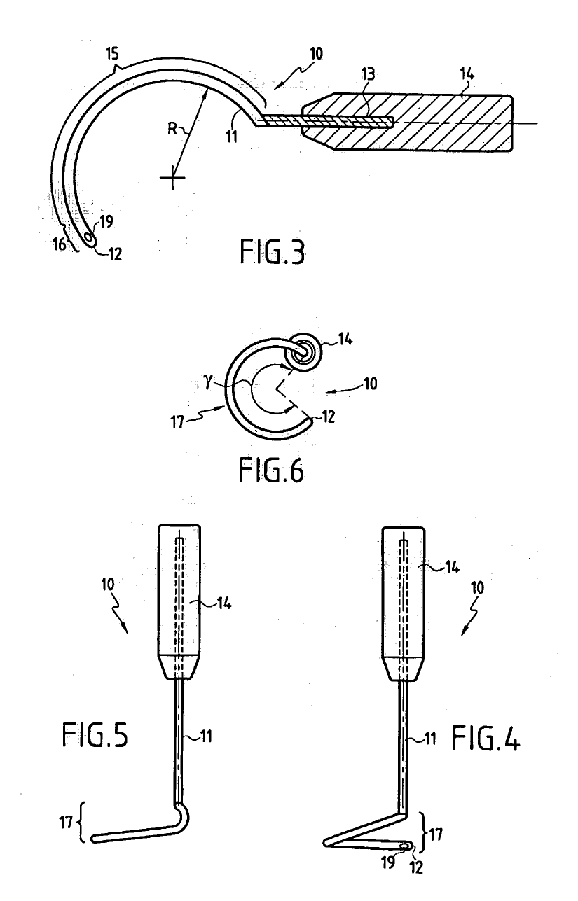

FIG. 3 shows a partially torn out elevation of a perforator guide that can be used for placement of the implant according to the invention and with an arc shape.

FIG. 4 is an elevation of another embodiment of a perforator guide according to the invention, with a helical-shaped insertion end.

FIG. 5 is a left view of the perforator guide according to FIG. 4.

FIG. 6 is a bottom view of the perforator shown in FIG. 4.

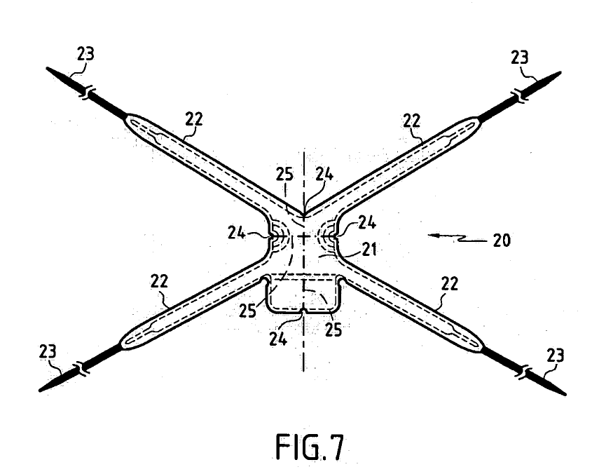

FIG. 7 is a view of an inserter according to the invention, used for placement of the implant shown in FIG. 1.

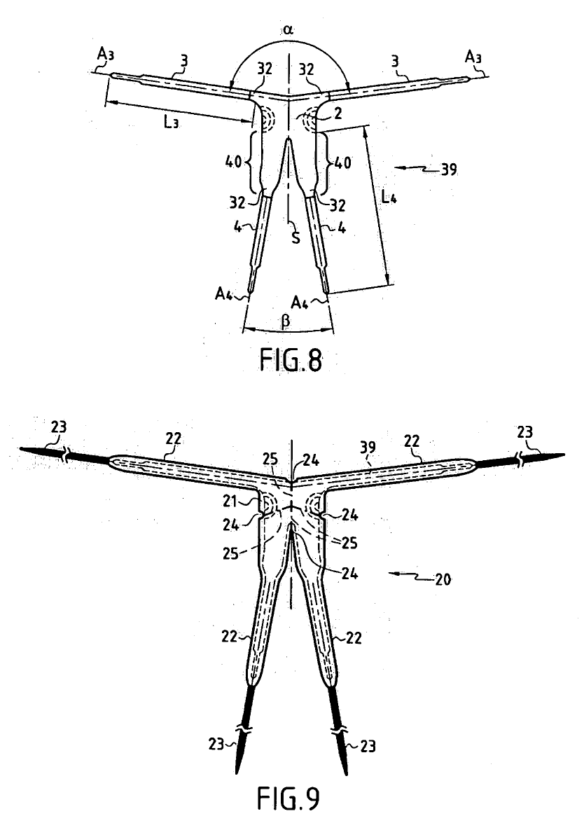

FIG. 8 is a view similar to FIG. 1 showing a variant embodiment of the implant according to the invention.

FIG. 9 is a view of an inserter according to the invention, used for placement of the implant shown in FIG. 8.

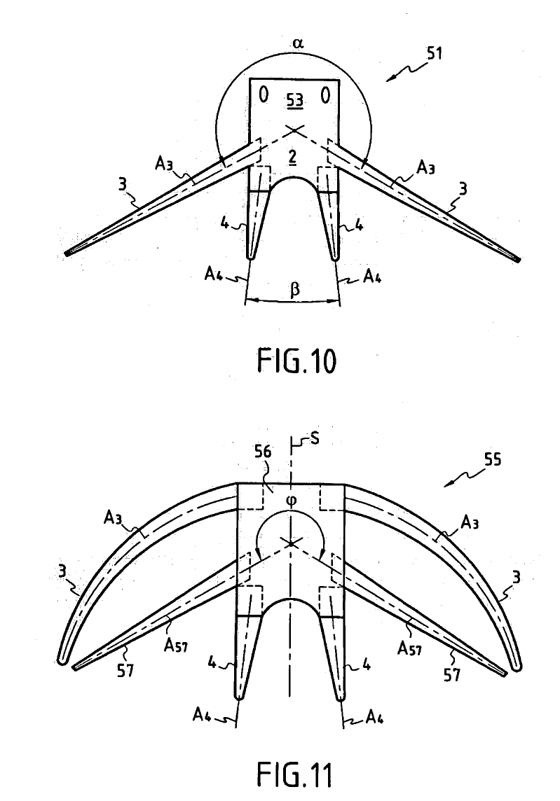

FIGS. 10 and 11 are views similar to FIG. 1, showing variant embodiments of an implant according to the invention.

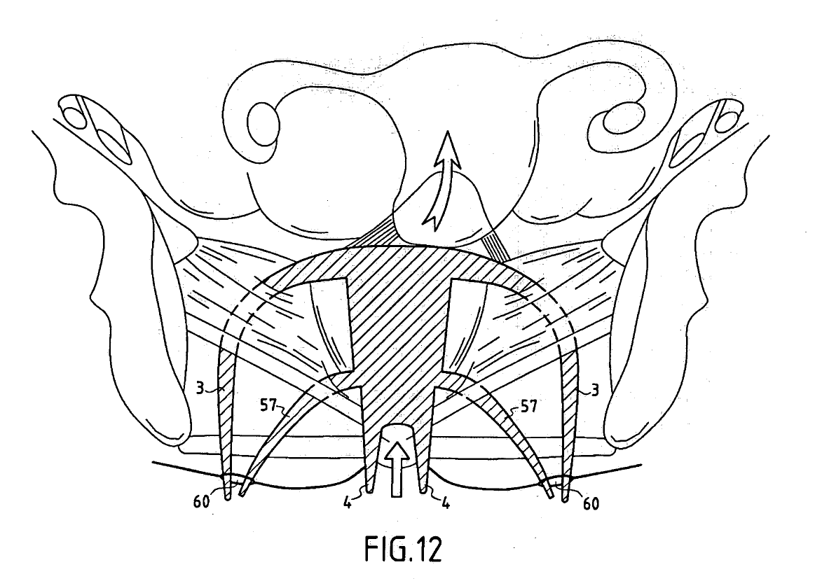

FIG. 12 shows an anatomic view showing an example of how an implant according to FIG. 11 is implanted in a woman.

The invention proposes an implant designed more particularly for the treatment of rectocele and denoted as a whole by reference 1 in FIG. 1. This implant 1 has a thin and flexible structure and is made from an adapted biocompatible material, for example such as woven or non-woven synthetic material or a knitted material based on polypropylene or polyester fibres. Such a synthetic material may or may not then be coated with products facilitating cell growth.

Similarly, the implant according to the invention may be made from natural materials such as “fascia latta” or any other absorbable biological or synthetic material.

According to the invention, the implant 1 comprises a support body 2 from which two suspension stabilisers 3 extend, arranged on each side of a sagittal plane S. The implant also comprises two lower suspension stabilisers 4 arranged on each side of the sagittal plane S, these stabilisers acting through the stricture made by the muscular masses.

In the example shown, the support body 2 has an approximately rectangular shape, although this shape cannot be considered as being strictly necessary for the invention, and the upper suspension stabilisers 3 each extend from an upper corner of the body 2. Each of the lower suspension stabilisers 4 extends from one side of the rectangular shaped support body 2.

Thus, considering the arrangement of the lower suspension stabilisers 4, there is a sort of small lower apron 9 at the body 2 of the implant 1. According to the example shown, the lower suspension stabilisers 4 initiate on a side of the support body 2 at a distance d from the upper edge 30 of the support body 2, preferably but not strictly necessary between 60% and 87% of the length L₂ of the support body.

Preferably, the support body 2 is chosen to have a length L₂ between 60 mm and 90 mm and a width 12 between 40 mm and 60 mm.

According to one preferred characteristic of the invention, the longitudinal axes A₃ of the upper stabilisers 3 form an angle α greater than 45°, and preferably between 100° and 180°, and ideally between 115° and 170°.

Furthermore, according to the example embodiment shown in FIG. 1, there is a non-zero angle β between the axes A₄ of the lower suspension stabilisers 4, the angle β preferably being greater than 10° and in this example embodiment between 100° and 180°. It should be noted that the sagittal plane S is preferably a plane of symmetry of the implant 1 and therefore bisects the angles α and β.

The lengths of the upper suspension stabilisers 3 and the lower suspension stabilisers 4 measured between the distal end of each suspension stabiliser and the support body 2 are L₃, L₄ respectively, preferably but not necessarily more than 100 mm and even better more than 120 mm, to facilitate placement of the suspension stabilisers in their corresponding insertion areas while providing an optimum friction surface area with the tissues passed through. In addition, the width of the suspension stabilisers is preferably but not exclusively between 5 mm and 15 mm and for example may be equal to about ten millimetres.

The suspension stabilisers are made so as to have low extensibility parallel to their longitudinal axis A₃, A₄, so as to avoid their deformation under the effect of tension applied to them during placement of the implant. In the embodiment shown, this result is achieved by making each of the stabilisers 3, 4 from a band of polyethylene knit in which the rows of stitches are oriented transverse to the longitudinal axis of each stabiliser. Each band from which the suspension stabiliser is made is then added on it and fixed to the support body using a seam 31 onto an attachment tab 32 forming part of the body 2. To facilitate understanding, FIG. 2 illustrates an exploded view of the implant before the suspension stabilisers 3, 4 are attached onto the body 2. It should be noted that in this example, the body 2 of the implant is made from a knit with looser and more extensible stitches than the knit from which the suspension stabilisers are made. Furthermore, the upper suspension stabilisers 3 and the lower suspension stabilisers 4 according to the invention are independent of each other and are only connected together through the body 2.

As described above, the implant 1 will be placed at the rectovaginal partition of a patient. To achieve this and to minimise dissection of this region and the resulting trauma, the invention proposes that the surgeon performing the treatment can use one or several elongated perforator guides 10, like those shown particularly in FIGS. 3 and 4 to 6.

In general, such a perforator guide 10 comprises an elongated body or mandrel 11 for which one end 12 will be introduced into the body of the patient to be treated and the other end 13 of which is provided with a handle 14. It should be noted that the insertion end 12 is preferably made from a foam tip, in other words a non-traumatic tip that will not injure or cut the tissues into which it is to be inserted.

According to one embodiment shown in FIG. 3, the perforator guide is arc-shaped in a plane. This arc shape in a plane is particularly suitable for placement of suspension stabilisers in transperineal and transgluteal areas. Preferably but not strictly necessary, the arc-shaped part of the perforator guide then has a radius of curvature R between 30 mm and 60 mm and preferably, for the part 15 of the perforator guide 10 extending between the handle 14 and the end 12, between 40 mm and 50 mm, the end part 16 of the perforator guide 10 then having a variable radius of curvature.

According to another embodiment of the perforator guide 10 shown in FIGS. 4 to 6, the elongated body 11 of the guide 10 has a helical-shaped end 17, also adapted for the placement of suspension stabilisers in the upper or lower area of closed off holes. Preferably, the distal end 17 of the perforator guide is then shaped like a portion of a helical turn extending over an angle γ between 180° and 360°, and preferably between 255° and 270°. Also preferably, the turn 17 of the perforator guide has a radius of curvature between 20 mm and 40 mm with a pitch between 15 mm and 25 mm.

The implant 1 according to the invention is preferably arranged so that there is no residual tension after it has been put into place for at least some of its suspension stabilisers. In one variant operation type, the invention proposes to facilitate this operational gesture by using an inserter, more particularly as shown in FIG. 7 and denoted as a whole by reference 20.

This inserter has a flexible structure and its shape is similar to the shape of the implant. The inserter 20 is preferably made from a biocompatible polymer material from the family of plastics with a low coefficient of friction, for example such as polyethylene. The inserter 20 then includes a hollow body 21 defining a cavity for housing the body 2 of the implant 1. The inserter 20 also comprises tubular stabilisers 22 that extend from the hollow body 21, with each defining a cavity for housing a suspension stabiliser 3, 4 and 5 of the implant 1. Each tubular strap 22 then has tension means 23 extending from the free end of the corresponding strap 22. The tension means 23 may be made in any appropriate manner, for example by systems for fastening the ends of the stabilisers 22 onto a perforator guide 10. According to the example shown in FIG. 7, the tension means 23 comprise a flexible or semi-rigid needle for each strap 22, with a non-traumatic or foam end. Such a needle may be made from the same material as the material from which the inserter 20 is made, or more generally a material chosen from among synthetic polymers preferably with a low coefficient of friction.

Finally, the inserter 20 includes cutting means 24, the function of which will become clear later, for cutting at least the hollow body 21 in the inserter 20. The cutting means 24 may then be made in any appropriate manner, and in the example shown include a series of six openings 24 made around the periphery of the hollow body 21 between each of the tubular stabilisers 22, to enable a cutting tool to pass through to cut the hollow body 21 along the lines 25 materialised by the chained dotted lines in FIG. 7.

The implant 1 is arranged inside the hollow body 21 and the tubular stabilisers 22, and is preferably free inside these stabilisers such that the forces applied on the inserter 20 are not transmitted to the implant 1 itself.

The surgical treatment of rectocele using an implant 1 and perforator guides 10 as described above is applied as follows.

The patient to be treated is firstly anaesthetized, either generally or regionally or locally depending on the preferences of the surgeon and the condition of the patient's health. The operating position of the patient on the operating table will be the position usually adopted for vaginal surgery, in other words the patient's buttocks slightly beyond the operation table and the thighs moderately bent onto the abdomen.

Firstly, a rectal mesh is put into place and an ischemic injection is made.

The area that will receive the support body 2 of the implant 1 is then dissected. Tension is applied to the cervix uteri, to expose the posterior formix of vagina. A vaginal incision is made on the posterior part of the cervix uteri, transversely on the cervical side of the formix of vagina clearly exposed by the tension. This incision may be qualified as horizontal retrocervical. The lower vaginal section edge thus made is gripped entirely by three Alis clips that are pulled downwards to expose the recto-vaginal plane.

A recto-vaginal separation is then made progressively, releasing the posterior vaginal wall. This separation is stopped at the bottom and in the middle above the anal cap. The separation is continued laterally along the dissection plane and in contact with the levator muscle passing (from bottom to top) through the pubo-coccygien bundle, then the ilio-coccygien bundle to reach the coccygien muscle and the sacrosciatic ligament. This dissection is carried out cautiously, pushing the peri-rectal fat and the rectum inwards. It should be noted that no other structure needs to be sectioned and that the peri-rectal fat can be pushed inwards while remaining in contact with the muscular floor. The most practical method of doing this dissection consists of using a finger, a compress and a set of narrow and long vaginal blades (modified Breisky blades), because they enable progressive controlled opening of the view of the space without excessive enlargement allowing a finger to be inserted for execution of the dissection.

Once this dissection work has been done, the implant 1 can be inserted, either bare or encapsulated in the inserter 20.

The first step is to place a first upper suspension stabiliser 3. This is done by using an arched perforator guide 10 chosen by the surgeon, like that described previously with reference to FIGS. 3 and 4 to 6. The perforator guide 10 is then led through the patient's buttock by a puctiform incision located about 15 mm behind the median point of the line extending from the anus to the ischion. A finger inserted into the previously executed lateral separation at the contact of the sacro-iliac ligament will then receive the tip of the perforator guide so as to lead it into the lower vaginal incision. A tension element 23 of the inserter 20 cooperates with an upper suspension stabiliser 3, and is fixed in an eye 19 in the end of the perforator guide 10 and the perforator guide is withdrawn in the reverse direction so as to entrain the tubular strap 22 of the inserter 20 and the stabiliser or upper suspension arm 3 contained in it. The tensioned strap then passes through the sacrosciatic ligament. It must be mentioned that in the absence of an inserter 20, the distal end of the upper suspension stabiliser is fixed directly onto the perforator guide 10 so that it can be pulled.

The same gesture is made for placement of the second upper strap 3. The upper stabilisers 3 thus inserted for transgluteal suspension are then put in waiting on clips.

The lower suspension stabilisers 4 are then passed through the puborectal muscle on each side of the anal channel and are externalised by the same buttock orifice as the upper stabilisers 3.

Once the four suspension stabilisers 3, 4 have been engaged, the inserter 20 is cut out so as to release the implant 1. The removal of the different constituents of the inserter 20 by tensions applied in pairs on the opposite tubular stabilisers 22 is thus used to put the implant 1 into place, without any stress, such that the implant is in a state that can be qualified as being relaxed.

The support body 2 of the implant 1 is then fixed on the utero-sacral ligaments and the apron 9 is fixed on the lower face of the cervix uteri by one or several and preferably three absorbable sutures.

The posterior vaginal incision is then sutured by an absorbable thread and tension is then applied on the upper suspension stabilisers 3 passing through the sacrosciatic region in order to bring the vaginal dome back into its right position.

Any excess length of the upper suspension stabilisers 3 and the lower suspension stabilisers 4 can then be sectioned and the buttock orifices can be closed using sutures made from absorbable suturing thread.

A vaginal mesh is inserted at the end of the operation with a cystic probe that will be removed forty-eight hours after the operation. Post-urination residues will then be measured by catheterisation, to assure that the cystic drain is satisfactory and so that the patient can be released.

The operation for the treatment of rectocele will last about one hour and an average hospitalisation of four days should be allowed. The patient's activities will be restricted for a month and bathing should be avoided during this period. Finally, a period of six weeks sexual abstinence after the operation should be respected.

The previously proposed technique thus treats only the pathology, namely the unbalance of the pelvic statics and therefore makes the anatomy as normal as possible while maintaining the individual's body shape. Advantageously, this technique provides a means of keeping healthy organs or organs not having an unfavourable influence on the pelvic statics. A cancer pathology will have been eliminated in the pre-operating check-up and it will be possible to maintain reliable gynaecological monitoring after the surgery.

There are very low risks of pelvic genital cancer and the treatment proposed by the invention does not complicate subsequent access to the genitals and the rectal region.

According to the example embodiment of the implant shown in FIG. 1, the lower suspension stabilisers 4 are fixed on tabs 32 that extend from the sides of the support body, however this arrangement of the tabs is not strictly necessary for making an implant according to the invention. Thus, FIG. 8 shows an implant 39 according to the invention in which the four suspension stabilisers (two upper stabilisers 3 and two lower stabilisers 4) are added onto the tabs 32 that extend from the four corners of the support body 2. In this case, the suspension stabilisers are sutured at their far end onto the far end of the corresponding tab 32.

According to this embodiment, the axes A₃ of the upper suspension stabilisers form an angle α with the same characteristics as the implant 1 described above, while the axes A₄ of the lower suspension stabilisers 4 form an angle β preferably but not exclusively between 10° and 75°.

Moreover, the attachment tabs 32 of the lower suspension stabilisers 4 have a broader shape than the suspension stabiliser, so as to form a pre-rectal suspension area 40.

FIG. 8 illustrates an example inserter 20 adapted more particularly to conformation of the implant 39.

The operation for the treatment of rectocele using the implant 39 is carried out as described above concerning dissection of the recto-vaginal area in which the support body 2 is fitted, and placement of the upper suspension stabilisers 3 and the lower suspension stabilisers 4.

The broadened parts 40 of the lower suspension stabilisers 4 may be fixed to the levator muscles in the puborectal region by absorbable sutures.

Similarly, it will be possible for lower suspension stabilisers 4 to pass through the perineum. This pre-rectal hammock put into trans-perineal position advantageously fixes the perineal plane with the upper transgluteal suspension so as to reinforce descended perinea.

According to the example implant embodiments described above, the upper and lower suspension stabilisers 3 and 4 extend from the body of the implant while diverging so that the implant has a shape that could be called a star. However, this star-shape is not strictly necessary for making an implant according to the invention, and for some pathologies it may be necessary to use an implant for which the suspension stabilisers do not diverge but on the contrary for which the free ends of the upper stabilisers 3 and the lower stabilisers 4 face downwards. In such a case, the longitudinal axes A₃ of the upper stabilisers then form an angle α greater than 180° and preferably greater than 200°.

FIG. 13 shows such an embodiment of an implant 51 according to the invention in which the upper stabilisers 3 extend from the sides of the body 52 of the implant at a distance of its upper edge while the two lower stabilisers 4 extend from the lower edges of the support body 52. This particular configuration then defines a sort of upper apron 53 provided with two orifices 54 for passage of an add-on stabilisation band or for the passage of posterior stabilisers of an anterior prosthesis used in association with the implant according to the invention, once the stabilisers of the said anterior prosthesis have trans-fixed the utero-sacral ligaments.

Moreover, according to the embodiment shown in FIG. 13, the axes A₃ of the upper suspension stabilisers 3 form an angle α particularly between 210° and 260° while the axes A₄ of the lower suspension stabilisers 4 form an angle α less than 45° and preferably zero.

It should be noted that according to this embodiment, the body 2 of the implant does not include a specific tab for attachment of suspension stabilisers that in this case are bonded directly onto the support body.

FIG. 11 illustrates yet another variant embodiment of an implant 55 for the treatment of prolapse of the vaginal dome and rectocele, in which the suspension stabilisers are all facing downwards. In this variant, the body 56 of the implant is rectangular in shape. The two upper suspension stabilisers 3 and the two lower suspension stabilisers 4 then extend from the upper and lower corners respectively of the support body 56. According to this example, the upper suspension stabilisers 3 are arc-shaped and their neutral axes A₃ that are the equivalent of axes A₃ in the case of an arm or straight stabiliser form an angle α more than 180°. The angle α is measured between two tangents of the neutral axes of the upper suspension stabilisers. It should be noted that the concaveness of the upper suspension stabilisers 3 faces downwards.

According to a variant embodiment shown in FIG. 11, the implant 55 also comprises middle suspension stabilisers 57 arranged on each side of the sagittal plane S and between the upper stabilisers 3 and the lower stabilisers 4. The ends of the middle suspension stabilisers face downwards and their axes A₅₇ form an angle φ preferably greater than 200° and ideally between 210° and 260°. Furthermore, the ends of the upper stabilisers 3 and the middle suspension stabilisers 57 located on the same side of the sagittal plane S converge towards a point or at least approximately in the same direction as shown in FIG. 14, this characteristic facilitating the implantation procedure as will be described later. According to this example, each of the stabiliser arms is welded directly onto the body of the implant.

Placement of the prosthesis as shown in FIG. 11 and described above for the treatment of the prolapse of the vaginal dome and rectocele then comprises a dissection phase after the patient has been anaesthetised. A Muze clip is then used that applies tension on the cervix uteri so as to expose the posterior formix of vagina. A vaginal incision is made on the posterior face of the cervix uteri transversely on the cervical slope of the formix of vagina clearly exposed by the tension.

The edge of the posterior vaginal section is gripped entirely by 3 Alis clips that are pulled downwards to expose the rectovaginal plane. The rectovaginal separation is carried out progressively, by moving the posterior vaginal wall outwards. This separation is stopped at the bottom and in the middle above the anal cap. There is absolutely no point in making a dissection of the recto anal plane, and it would even be harmful since this is a non-anatomic plane obviously created by surgery. Laterally, the dissection plane is in contact with the levator muscle, passing from bottom to top through the pubo-coccygien bundle then the ilio-coccygien bundle to reach the coccygien muscle and the sacrosciatic ligament. This dissection is carried out cautiously, pushing the perirectal fat and the rectum inwards. No structure has to be sectioned; all that is necessary is to push the perirectal fat inwards while remaining in contact with the muscular floor. The most practical method is to use a finger, a compress and particularly a set of two long and narrow vaginal blades; these progressively open the space under visual control without excessive enlargement of the dissected space, but sufficient for a finger to enter.

Finally, a perforator guide 10 is passed through the buttock by a punctiform incision 60 located 15 mm behind the median point of the line extending from the anus to the ischion as shown in FIG. 12. The finger inserted into the lateral separation in contact with the sacro-iliac ligament will hold the tip of the perforator guide 10 and will lead it into the posterior vaginal incision, one end of an upper stabiliser 3 of the implant is fixed on the perforator guide 10 and is pulled through the sacrosciatic ligament (sacrosciatic suspension), and the same action is taken on the other side for the other upper stabiliser of the implant 55. The upper edge of the implant is then sutured to the posterior face of the isthmus and to the uterosacral ligaments by 2 to 4 stitches. The two free ends of the upper stabilisers 3 of the implant are then put in waiting on the clip. The middle suspension stabilisers 57, called the puborectal suspension stabilisers, are passed through the puborectal muscles by a transperineal channel using the same posterior entry orifice as for the upper suspension stabilisers but with an inwards orientation to emerge on average at ⅓ of the height of the colpocele through the puborectal muscle. The lower suspension stabilisers, also called perineal stabilisers, are passed on each side of the vulvar fork through a perforator guide 10 that is better inserted from the inside towards the outside (top to bottom) rather than from bottom to top, since in this direction the instrument moves towards the rectum and it could be a threat to it.

At the end of the operation, the posterior vaginal incision is sutured by absorbable thread and a medium tension is applied to the upper and middle suspension stabilisers to lift the vaginal dome to the right position and to put the pre rectal hammock into position without excessive tension.

The operation is then completed in the same way as was described above.

1. Implant for the treatment of cystocele, rectocele and/or prolapse of the vaginal dome, with a thin, flexible structure including a support (2) body starting from which extend at least:
two upper suspension stabilisers (3),
and two lower suspension stabilisers (4) on each side of the sagittal plane (S),
characterised in that each suspension stabiliser (3, 4, 57) is made so that its extensibility parallel to its longitudinal axis is low.

2. Implant according to claim 1, characterised in that the extensibility of suspension stabilisers (3, 4, 57) parallel to their longitudinal axis is less than the extensibility of the body of the implant particularly along its longitudinal axis and its transverse axis.

3. Implant according to claim 1, characterised in that each suspension stabilizer (3, 4, 57) is added onto the support body (2).

4. Implant according to claim 3, characterised in that the support body has an attachment tab (32) for each suspension stabiliser (3, 4).

5. Implant according to claim 3, characterised in that each suspension stabiliser (3, 4) is sewn onto the support body.

6. Implant according to claim 3, characterised in that each suspension stabiliser is glued onto the support body (2).

7. Implant according to claim 3, characterised in that each suspension stabiliser (3, 4) is welded onto the support body (2).

8. Implant according to claim 1, characterised in that each suspension stabiliser (3, 4, 57) is made from a knitted material for which the weft threads or the warp threads are arranged parallel to the longitudinal axis of the suspension stabiliser.

9. Implant according to claim 1, characterised in that each suspension stabiliser (3, 4, 57) is made from a slightly extensible knit parallel to the longitudinal axis of the suspension stabiliser.

10. Implant according to claim 1, characterised in that each suspension stabiliser (3, 4, 57) is made from a layer of non-woven material with an isotropic tensile strength in its extension plane.

11. Implant according to claim 1, characterised in that each suspension stabiliser (3, 4, 57) is made from a biocompatible synthetic material.

12. Implant according to claim 1, characterised in that each suspension stabiliser (3, 4, 57) is made from an absorbable or non-absorbable biocompatible material.

13. Implant according to claim 1, characterised in that the support body (2) has an approximately rectangular shape.

14. Implant according to claim 13, characterised in that the support body (2) has a length (L) between 60 mm and 90 mm and a width between 40 mm and 60 mm.
